(19) United States
(12) Patent Application Publication
(10) Pub. No.: US 2012/0125347 A1
(43) Pub. Date: May 24, 2012

(54) Bodysuit type garment for selective swaddling of an infant

(76) Inventor: Bree Ana Soileau, San Antonio, TX (US)

(21) Appl. No.: 12/927,820

(22) Filed: Nov. 24, 2010

(51) Int. Cl. A61F 5/37; A41D 11/00

(52) U.S. Cl. 128/873; 2/80

(57) ABSTRACT

A garment for selective swaddling of an infant includes an upper body portion for enclosing the torso region of the infant, a pair of sleeves extending outward from the upper body portion and crotch portion. Each of the outwardly extending sleeves is selectively closable such that a respective arm of the infant is precluded from passage therethrough and the crotch portion is selectively openable such that access is thereby provided to the interior of the garment. The upper body portion is provided with a selectively closable vertical opening extending downward from a collar of the upper body portion across a front portion of the upper body portion, thereby providing access into the upper body portion for tucking the infant's arms therein. A zipper is provided for selective opening and closing of the vertical opening. Snaps are provided for selective closure of the sleeves and selective opening of the crotch portion.

### FIELD OF THE INVENTION

The present invention relates to infant care. More particularly, the invention relates to an article of apparel specifically adapted to selectively convert between a swaddling garment and an otherwise ordinary and aesthetically pleasing outer garment.

### BACKGROUND OF THE INVENTION

Dating back thousands of years, the practice of snuggly wrapping infants in cloths or bands—known as swaddling—is thought to have originated in the belief that the practice was necessary for an infant to develop proper posture. Although swaddling was largely abandoned in the seventeenth century, the practice has recently resurged in the wake of medical studies indicating that swaddling assists infants in remaining in restful sleep by preventing the rooting reflex, wherein the infant attempts to suckle fingers coming into contact with his or her face and mouth, and, additionally that swaddling appears to reduce the risk of sudden infant death syndrome (“SIDS”). While these benefits surely suggest that swaddling should be wholeheartedly embraced, the matter is not so simply resolved.

In the first instance, swaddling presents the risk that the infant will become overheated if the ambient temperature is too high or the material is too thick, which, in turn, is thought to actually increase the risk of SIDS. While looser wrappings that are tucked into place as opposed to being tied can often be kicked off by a waking infant, allowing the infant to cool down, such loose wrappings may present a suffocation hazard unless some measure is taken to ensure that the swaddling article is prevented from covering the infant's face. As a result, proper swaddling generally takes a significant length of time, making diaper checks and changes more arduous than otherwise necessary and increasing the chance of completely waking a sleeping infant.

Additionally, proper temperature regulation generally requires that any other garment worn by the infant be removed prior to wrapping. As a result, an infant falling into sleep while dressed up is often wakened during the change from nicer clothing into a swaddling blanket, which, of course, generally defeats the purpose of swaddling.

With the foregoing shortcomings and disadvantages of the prior art in mind, it is therefore an object of the present invention to provide an article of apparel for swaddling of an infant that improves over the prior art by being much simpler to use.

Additionally, it is an object of the present invention to provide such an article of apparel that is also inherently less susceptible to causing overheating of a swaddled infant.

Still further, it is an object of the present invention to provide such an article of apparel that also includes an integral measure to prevent covering of the infant's face by any portion of the swaddling article, thereby eliminating the suffocation hazard presented by prior art methods and articles.

It is also an object of the present invention to provide such an article of apparel that also facilitates rapid and quiet diaper checks and/or changes, thereby making easier the caregiver's task as well as minimizing the chance that a sleeping infant will be unnecessarily awakened.

It is also an object of the present invention to provide such an article of apparel that is also aesthetically pleasing, doubling as an outer garment fit for wear on any occasion, in which an infant may be selectively swaddled.

Finally, it is an object of the present invention to provide such an article of apparel that is also simple and economical to manufacture, thereby being readily available for the widest use.

### SUMMARY OF THE INVENTION

In accordance with the foregoing objects, the present invention—a garment for the selective swaddling of an infant—generally comprises an upper body portion adapted to enclose the torso region of the infant, a pair of sleeves extending outward from the upper body portion and crotch portion. In critical aspects of the present invention, each of the outwardly extending sleeves is selectively closable such that a respective arm of the infant is precluded from passage therethrough and the crotch portion is selectively openable such that access is thereby provided to the interior of the garment. In an also critical aspect of the present invention, the upper body portion is provided with a selectively closable vertical opening extending downward from a collar of the upper body portion across a front portion of the upper body portion, thereby providing access into the upper body portion for tucking the infant's arms therein.

In the preferred implementations of the present invention, a zipper is provided in the vertical opening for selective opening and closing thereof. On the other hand, the preferred implementations of the present invention contemplate the utilization of snaps for providing selective closure of the sleeves as well as for providing selective opening of the crotch portion. Although other fasteners may be employed, snaps are generally preferred to the extent that they are quick and easy to secure and release and, additionally, are generally less noisy in operation than either releasably engageable hook and loop type fasteners and zippers as well as being generally more secure than sewn on buttons, the latter presenting a possible choking hazard.

In a first preferred implementation of the present invention, the crotch portion comprises an extension from the bottom end of the upper body portion, which extension may be formed as one or more flaps of garment material. In the exemplary embodiment comprising two flaps, the flaps are cooperatively adapted through size and shape to overlap one another to form the crotch portion. In this embodiment, a first one of the flaps is provided with a first set of snap halves and the second one of the flaps is provided with one or more matingly corresponding sets of snap halves. As will be appreciated by those of ordinary skill in the art, the provision of a second set of snap halves thereby provides adjustability to the crotch portion, such as may be particularly beneficial for accommodating a particularly long infant or very bulky diaper or other undergarment.

In a second preferred implementation of the present invention, the crotch portion comprises a pair of pant legs extending downward from the bottom end of the upper body portion. In this exemplary embodiment, the opening for the crotch portion is formed as a slit extending from the inseam of one pant leg to the inseam of the opposite pant leg. A first set of snap halves is provided adjacent the inseams of the pant legs at a first side of the thus formed selectively closable opening and one or more sets of snap halves matingly corresponding to the first set of snap halves is provided adjacent the inseams of the pant legs at a second side of said selectively closable opening of said crotch portion. As with the first described preferred implementation, those of ordinary skill in the art will recognize that the provision a second set of snap halves adjacent the second side of the opening provides this embodiment with the desired adjustability in the crotch portion.

Finally, many other features, objects and advantages of the present invention will be apparent to those of ordinary skill in the relevant arts, especially in light of the foregoing discussions and the following drawings, exemplary detailed description and appended claims.

### BRIEF DESCRIPTION OF THE DRAWINGS

Although the scope of the present invention is much broader than any particular embodiment, a detailed description of the preferred embodiment follows together with illustrative figures, wherein like reference numerals refer to like components, and wherein:

FIG. 1 shows, in a plan view, a first preferred implementation of the garment of the present invention;

FIG. 2 shows, in a side perspective view, the garment of FIG. 1 as worn by an infant in a first, ordinary garment configuration;

FIG. 3 shows, in a side perspective view corresponding to that of FIG. 2, the garment of FIG. 1 as worn by an infant in a second, swaddling garment configuration;

FIG. 4 shows, in an end perspective view, the garment of FIG. 1 as worn by an infant in the second, swaddling garment configuration of FIG. 3 and particularly depicting an implemented provision for access while the infant is swaddled to an undergarment additionally worn by the infant; and

FIG. 5 shows, in a plan view, a second preferred implementation of the garment of the present invention.

### DETAILED DESCRIPTION OF THE PREFERRED EMBODIMENT

Although those of ordinary skill in the art will readily recognize many alternative embodiments, especially in light of the illustrations provided herein, this detailed description is exemplary of the preferred embodiment of the present invention, the scope of which is limited only by the claims appended hereto.

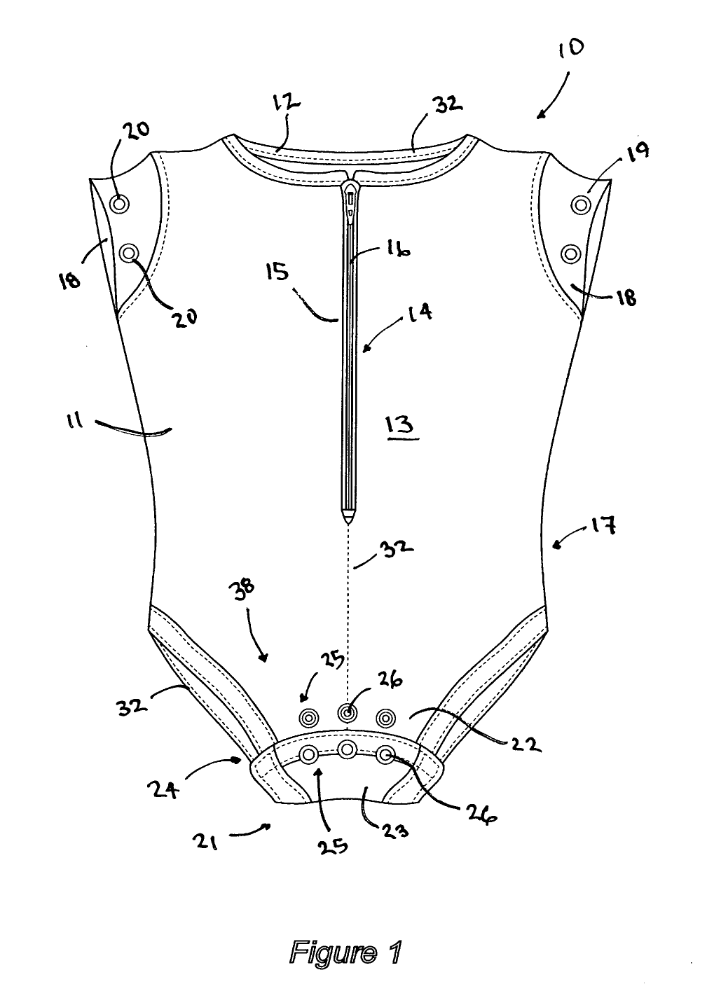

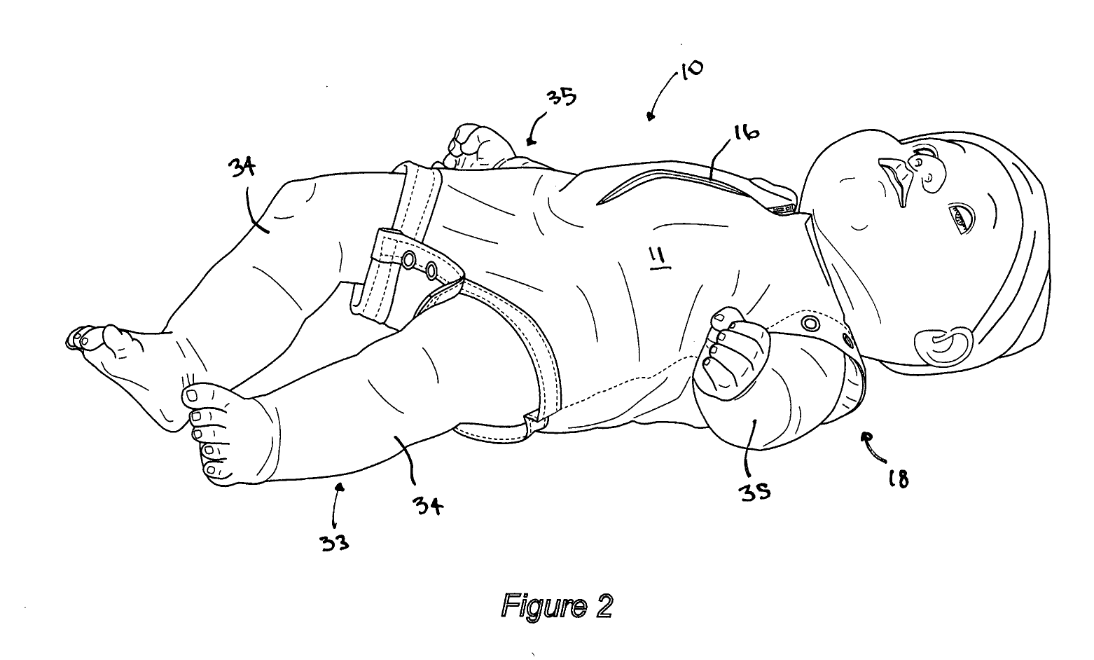

Referring now to the figures, and to FIGS. 1 and 2 in particular, a first preferred implementation of a bodysuit type garment 10 for the selective swaddling of an infant 33 is shown to generally comprise an upper body portion 11 adapted to enclose the torso region of the infant 33, a pair of sleeves 18 extending outward from the upper body portion 11 and crotch portion 38. In critical aspects of the present invention, each of the outwardly extending sleeves 18 is selectively closable such that a respective arm 35 of the infant 33 is precluded from passage therethrough and the crotch portion 38 is selectively openable such that an access opening 24, 29 is thereby provided to the interior of the garment 10. In an also critical aspect of the present invention, the upper body portion 11 is provided with a selectively closable vertical opening 14 extending downward from a collar 12 of the upper body portion 11 across a front portion 13 of the upper body portion 11, thereby providing access into the upper body portion 11 for tucking the infant's arms 35 therein.

In the preferred implementations of the present invention, a fastener 15, preferably comprising a zipper 16, is provided in the vertical opening 14 for selective opening and closing thereof. On the other hand, the preferred implementations of the present invention contemplate the utilization of snaps 20, 26, 31 for providing selective closure of the sleeves 18 as well as for providing selective opening of the crotch portion 38. Although other fasteners 19, 25, 30 may be employed, snaps 20, 26, 31 are generally preferred to the extent that they are quick and easy to secure and release and, additionally, are generally less noisy in operation than either releasably engageable hook and loop type fasteners and zippers as well as being generally more secure than sewn on buttons, the latter presenting a possible choking hazard.

In a first preferred implementation of the present invention, as particularly shown in FIG. 1, the crotch portion 38 comprises an extension 21 from the bottom end 17 of the upper body portion 11, which extension 21 may be formed as one or more flaps 22, 23 of garment material. In the exemplary embodiment comprising a two flaps 22, 23, the flaps 22, 23 are cooperatively adapted through size and shape to overlap one another to form the crotch portion 38. In this embodiment, a first one 25 of the flaps is provided with a first set of snap halves and the second one 26 of the flaps is provided with one or more matingly corresponding sets of snap halves. As will be appreciated by those of ordinary skill in the art, the provision of a second set of snap halves thereby provides adjustability to the crotch portion 38, such as may be particularly beneficial for accommodating a particularly long infant 33 or very bulky diaper 37 or other undergarment 36.

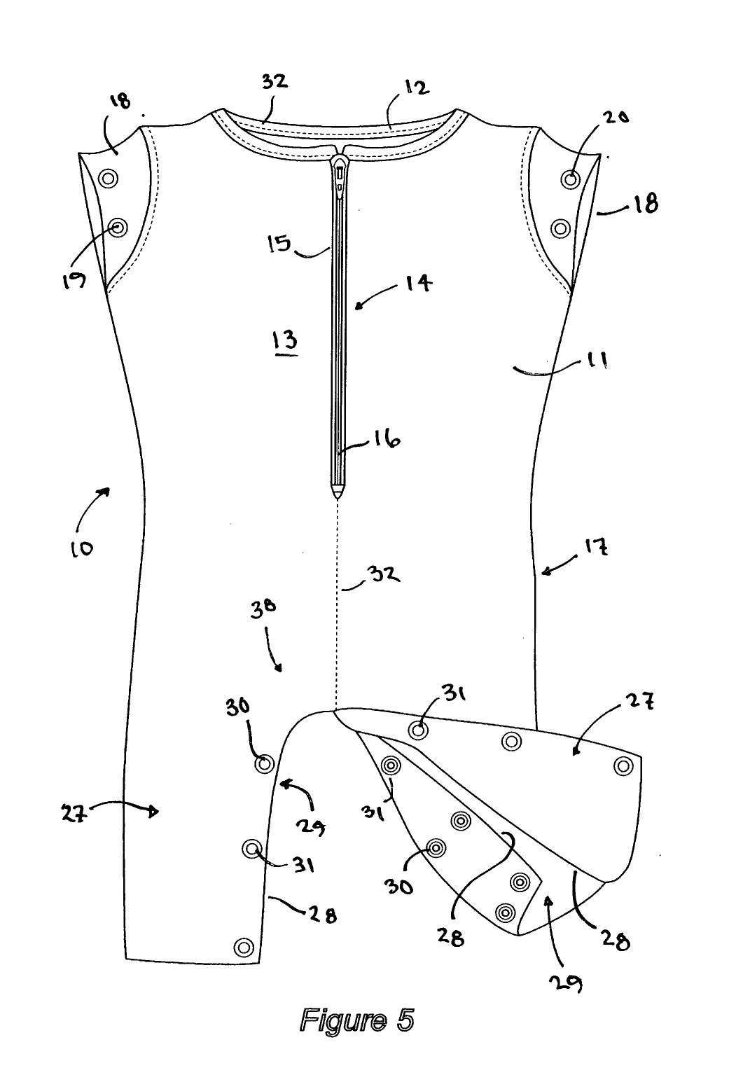

In a second preferred implementation of the present invention, as particularly shown in FIG. 5, the crotch portion 38 comprises a pair of preferably integral pant legs 27 extending downward from the bottom end 17 of the upper body portion 11. In this exemplary embodiment, the opening 29 for the crotch portion 38 is formed as a slit extending from the inseam 28 of one pant leg 27 to the inseam 28 of the opposite pant leg 27. A first set of snap halves is provided adjacent the inseams 28 of the pant legs 27 at a first side of the provided selectively closable opening 29 and one or more sets of snap halves matingly corresponding to the first set of snap halves is provided adjacent the inseams 28 of the pant legs 27 at a second side of said selectively closable opening 29 of said crotch portion 38. As with the first described preferred implementation, those of ordinary skill in the art will recognize that the provision a second set of snap halves adjacent the second side of the opening 29 provides this embodiment with the desired adjustability in the crotch portion 38.

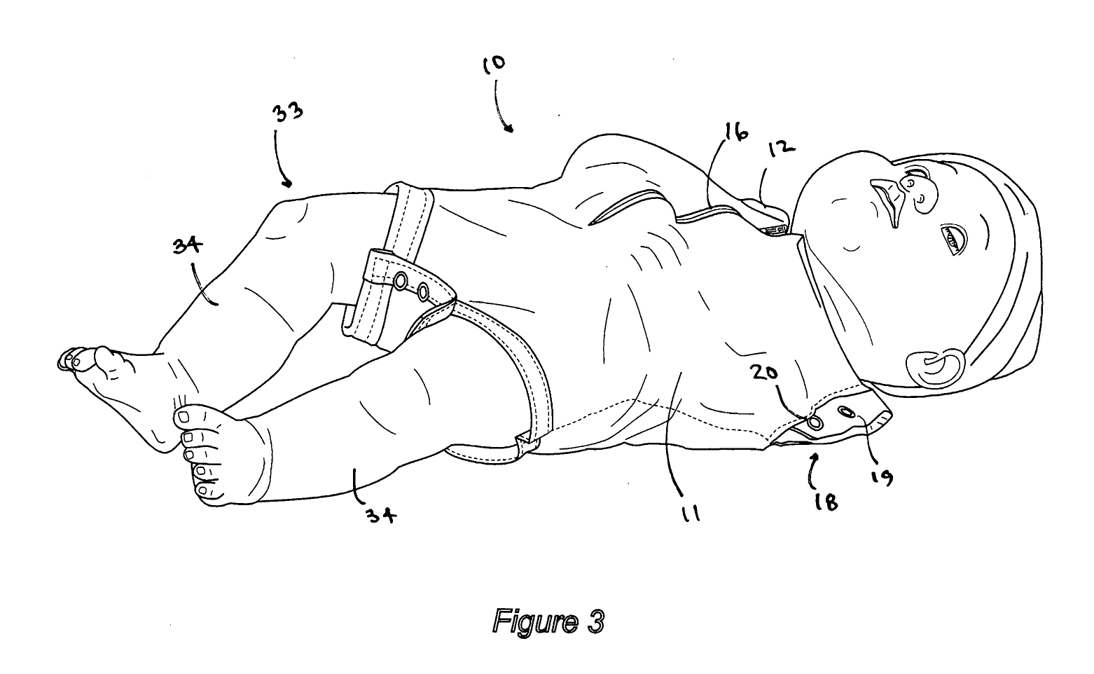

Referring now to FIG. 2, in particular, it is seen that the bodysuit type garment 10 of the present invention serves during the infant's waking hours as an ordinary appearing, aesthetically pleasing outfit, of which part the provision of the sleeves 18 is an important factor. As shown in FIG. 3, however, as the infant 33 falls off or is put down to sleep, his or her arms 35 may be readily tucked within the upper body portion 11 of the garment 10 by opening the zipper 16 at the front side 13 thereof and pulling his or her arms 18 through the open sleeves 18. The sleeves may then be closed with the provided fasteners 19 and the zipper 16 again closed, whereafter the infant 33 swaddled.

In addition to the aesthetic benefits of the provided sleeves 33, it is noted at this juncture that the provision thereof also has the added important benefit of providing room for the infant 33 to move to the extent necessary for comfort as well as necessary room to prevent overheating of the infant 33. To this end, it is noted that in at least one implementation of the present invention, the snap halves on a first portion of the sleeves 18 may be spaced differently than is the mating snap halves, thereby tending to form open spaces therebetween in facilitation of airflow therethrough.

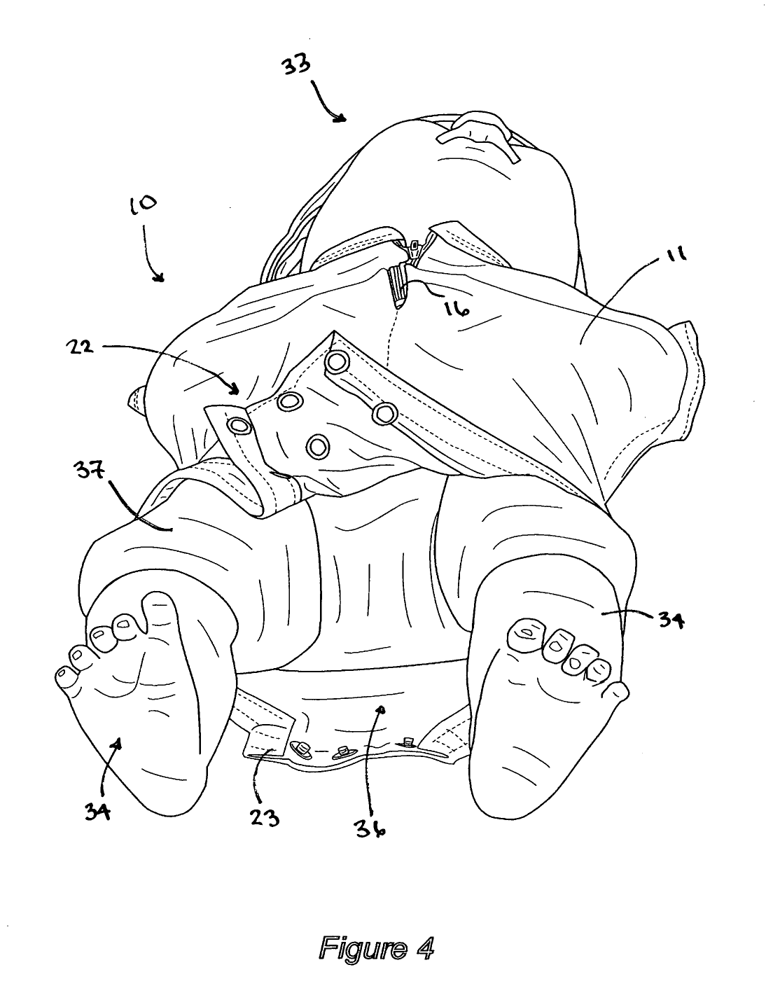

Finally, it is noted that it is considered critical to the present invention that the garment 10 is provided with a crotch portion 39 in order to prevent the garment from migrating upward and over or about the infant's face, which could cause a suffocation hazard or cause the infant 33 to overheat. As a result, as particularly shown in FIG. 4, it is deemed important to provide access into the garment 10 in the crotch region 38 in order that diaper checks or changes may be readily had without waking or unswaddling the infant 33. FIG. 4 shows, in particular, the manner in which the implementation of FIG. 1 accomplishes this objective.

While the foregoing description is exemplary of the preferred embodiment of the present invention, those of ordinary skill in the relevant arts will recognize the many variations, alterations, modifications, substitutions and the like as are readily possible, especially in light of this description, the accompanying drawings and claims drawn thereto. For example, those of ordinary skill in the art will recognize that the garment 10 may be constructed within the teachings of the present invention in any of a virtually unlimited forms using otherwise ordinary methods of manufacture such as, for example, stitching 32 or the like.

Likewise, those of ordinary skill in the art will recognize that, unlike prior art methods and articles for swaddling, the teachings of the present invention, which generally contemplate leaving the infant's legs 34 free to move, facilitate use of car seats, swings and the like. Additionally, the embodiment of FIG. 1 also leaves the infant's legs 34 free of the garment 10, thereby further ensuring that the infant 33 will not become overheated. In any case, because the scope of the present invention is much broader than any particular embodiment, the foregoing detailed description should not be construed as a limitation of the scope of the present invention, which is limited only by the claims appended hereto.

What is claimed is:

1. A garment for the selective swaddling of an infant, said garment comprising:
an upper body portion adapted to enclose the torso region of the infant, said upper body portion comprising:
a collar, said collar being located at a first, top end of said upper body portion; and
a selectively closable vertical opening, said vertical opening extending downward from said collar across a front portion of said upper body portion;
a pair of sleeves, each said sleeve being located substantially adjacent said collar at generally opposite sides thereof and extending outward from said upper body portion;
a crotch portion, said crotch portion being located at a second, bottom end of said upper body portion generally opposite said first, top end; and
wherein:
each said sleeve is selectively closable such that a respective arm of the infant is precluded from passage therethrough; and
said crotch portion is selectively openable such that access is thereby provided to the interior of said garment.

2. The garment for the selective swaddling of an infant as recited in claim 1, wherein said upper body portion further comprises a zipper for selectively closing said vertical opening.

3. The garment for the selective swaddling of an infant as recited in claim 1, wherein each said sleeve comprises a snap for the selective closing thereof.

4. The garment for the selective swaddling of an infant as recited in claim 1, wherein each said sleeve comprises a plurality of snaps for the selective closing thereof.

5. The garment for the selective swaddling of an infant as recited in claim 4, wherein each said plurality of snaps comprises:
a first set of snap halves arranged along a first region of the respective sleeve; and
a second set of snap halves arranged along a second region of the respective sleeve, each snap half of said second set being adapted for mating engagement with a snap half of said first set.

6. The garment for the selective swaddling of an infant as recited in claim 5, wherein the distance between snap halves of said second set differs from the distance between snap halves of said first set.

7. The garment for the selective swaddling of an infant as recited in claim 4, wherein said upper body portion further comprises a zipper for selectively closing said vertical opening.

8. The garment for the selective swaddling of an infant as recited in claim 4, wherein said crotch portion comprises an extension from said second, bottom end of said upper body portion.

9. The garment for the selective swaddling of an infant as recited in claim 8, wherein said extension comprises a flap.

10. The garment for the selective swaddling of an infant as recited in claim 9, wherein said extension comprises a first flap and a second flap, said first and second flaps being cooperatively adapted to overlap one another to form said crotch portion.

11. The garment for the selective swaddling of an infant as recited in claim 10, wherein said first flap is provided with a first set of snap halves.

12. The garment for the selective swaddling of an infant as recited in claim 11, wherein said second flap is provided with a second set of snap halves matingly corresponding to said first set of snap halves provided on said first flap.

13. The garment for the selective swaddling of an infant as recited in claim 11, wherein said upper body portion further comprises a zipper for selectively closing said vertical opening.

14. The garment for the selective swaddling of an infant as recited in claim 12, wherein said second flap is further provided with a third set of snap halves matingly corresponding to said first set of snap halves provided on said first flap, said third set of snap halves being offset a distance from said second set of snap halves to provide adjustability for said crotch portion.

15. The garment for the selective swaddling of an infant as recited in claim 14, wherein said upper body portion further comprises a zipper for selectively closing said vertical opening.

16. The garment for the selective swaddling of an infant as recited in claim 4, wherein:
said crotch portion comprises a pair of pant legs extending downward from said second, bottom end of said upper body portion; and
a selectively closable opening extending from the inseam of one said pant leg to the inseam of the other said pant leg.

17. The garment for the selective swaddling of an infant as recited in claim 16, wherein:
a first set of snap halves is provided adjacent the inseams of said pant legs at a first side of said selectively closable opening of said crotch portion; and
a second set of snap halves matingly corresponding to said first set of snap halves is provided adjacent the inseams of said pant legs at a second side of said selectively closable opening of said crotch portion.

18. The garment for the selective swaddling of an infant as recited in claim 17, wherein said upper body portion further comprises a zipper for selectively closing said vertical opening.

19. The garment for the selective swaddling of an infant as recited in claim 17, wherein a third set of snap halves matingly corresponding to said first set of snap halves is provided adjacent the inseams of said pant legs at said second side of said selectively closable opening of said crotch portion, said third set of snap halves being offset a distance from said second set of snap halves to provide adjustability for said crotch portion.

20. The garment for the selective swaddling of an infant as recited in claim 19, wherein said upper body portion further comprises a zipper for selectively closing said vertical opening.
